SEQUENCE LISTING (11 sequences)

SEQ ID NO: 1 - DNA, 18 bp, synthetic construct
  source 1..18: mol_type = other DNA
tgagaactttgtccaggc

SEQ ID NO: 2 - DNA, 19 bp, synthetic construct
  source 1..19: mol_type = other DNA
gtgctggctccatcgacca

SEQ ID NO: 3 - DNA, 22 bp, synthetic construct
  source 1..22: mol_type = other DNA
cacacaacatgtaaaccaggga

SEQ ID NO: 4 - DNA, 19 bp, synthetic construct
  modified_base 7: mod_base = OTHER; note = uracil
  source 1..19: mol_type = other DNA
aaggagtacaagcgaggaa

SEQ ID NO: 5 - DNA, 19 bp, synthetic construct
  source 1..19: mol_type = other DNA
  modified_base 6: mod_base = OTHER; note = uracil
  modified_base 10: mod_base = OTHER; note = uracil
  modified_base 12: mod_base = OTHER; note = uracil
ccacatcagtatccgacaa

SEQ ID NO: 6 - DNA, 19 bp, synthetic construct
  source 1..19: mol_type = other DNA
  modified_base 2: mod_base = OTHER; note = uracil
  modified_base 10: mod_base = OTHER; note = uracil
  modified_base 18: mod_base = OTHER; note = uracil
  modified_base 19: mod_base = OTHER; note = uracil
ctaaggagatggaccgctt

SEQ ID NO: 7 - DNA, 19 bp, synthetic construct
  source 1..19: mol_type = other DNA
  modified_base 6: mod_base = OTHER; note = uracil
  modified_base 7: mod_base = OTHER; note = uracil
  modified_base 10: mod_base = OTHER; note = uracil
  modified_base 12: mod_base = OTHER; note = uracil
agggattgatctcgagaac

SEQ ID NO: 8 - DNA, 19 bp, synthetic construct
  source 1..19: mol_type = other DNA
  modified_base 10: mod_base = OTHER; note = uracil
  modified_base 11: mod_base = OTHER; note = uracil
  modified_base 17: mod_base = OTHER; note = uracil
  modified_base 18: mod_base = OTHER; note = uracil
  modified_base 19: mod_base = OTHER; note = uracil
ggacgagccttcaaacttt

SEQ ID NO: 9 - DNA, 19 bp, synthetic construct
  source 1..19: mol_type = other DNA
  modified_base 4: mod_base = OTHER; note = uracil
  modified_base 10: mod_base = OTHER; note = uracil
  modified_base 18: mod_base = OTHER; note = uracil
gcatccacgtgcaaaggta

SEQ ID NO: 10 - DNA, 19 bp, synthetic construct
  source 1..19: mol_type = other DNA
  modified_base 13: mod_base = OTHER; note = uracil
  modified_base 15: mod_base = OTHER; note = uracil
  modified_base 17: mod_base = OTHER; note = uracil
gcaggaacgccgtgtctca

SEQ ID NO: 11 - DNA, 19 bp, synthetic construct
  source 1..19: mol_type = other DNA
  modified_base 1: mod_base = OTHER; note = uracil
  modified_base 3: mod_base = OTHER; note = uracil
  modified_base 7: mod_base = OTHER; note = uracil
  modified_base 9: mod_base = OTHER; note = uracil
  modified_base 10: mod_base = OTHER; note = uracil
  modified_base 18: mod_base = OTHER; note = uracil
tatcagtattcgccaacta

(19) United States
(12) Patent Application Publication
(10) Pub. No.: US 2026/0250684 A1
(43) Pub. Date: Aug. 27, 2026

(54) METHODS OF TREATING PANCREATIC CANCER

(71) Applicant: The Regents of the University of California, Oakland, CA (US)

(72) Inventor: Andrew M. Lowy, La Jolla, CA (US)

(21) Appl. No.: 19/161,390

(22) PCT Filed: Mar. 1, 2024
(86) PCT No.: PCT/US2024/018080

§ 371 Date: Sep. 2, 2025

Related U.S. Application Data
(60) Provisional application No. 63/488,380, filed on Mar. 3, 2023.

(51) Int. Cl. C12N 15/113; A61K 31/255; A61K 31/282; A61K 31/337; A61K 31/4745; A61K 31/475; A61K 31/513; A61K 31/5685; A61K 31/573; A61K 31/664; A61K 31/704; A61K 31/7105; A61P 35/04

(52) U.S. Cl. CPC C12N 15/1137; A61K 31/255; A61K 31/282; A61K 31/337; A61K 31/4745; A61K 31/475; A61K 31/513; A61K 31/5685; A61K 31/573; A61K 31/664; A61K 31/704; A61K 31/7105; A61P 35/04; C12N 2310/14; C12N 2310/531

(57) ABSTRACT

Provided herein are compositions and methods of treating pancreatic cancer in a subject that include: administering to the subject a therapeutically effective amount of a composition comprising an inhibitor of MICAL2. Also provided herein compositions including an inhibitory nucleic acid of MICAL2, wherein the inhibitory nucleic acid comprises any one of SEQ ID NOs: 1-11.

### CROSS-REFERENCE TO RELATED APPLICATIONS

This application claims priority to U.S. Provisional Patent Application No. 63/488,380, filed on Mar. 3, 2023. The disclosure of the prior application is considered part of the disclosure of this application and is incorporated herein by reference in its entirety.

### FEDERALLY SPONSORED RESEARCH OR DEVELOPMENT

This invention was made with Government support under Grant No. CA273974 awarded by the National Institutes of Health. The Government has certain rights in the invention.

### SEQUENCE LISTING

This application contains a Sequence Listing that has been submitted electronically as an XML file named “Sequence_Listing.” The XML file, created on Mar. 1, 2024, is 28,672 bytes in size. The material in the XML file is hereby incorporated by reference in its entirety.

### BACKGROUND

Pancreatic ductal adenocarcinoma (PDAC) remains one of the most lethal malignancies worldwide, characterized by early metastasis, resistance to conventional therapies, and dismal prognosis. Despite notable progress in understanding the genetics of pancreatic ductal adenocarcinoma (PDAC), discerning transcriptional subtypes, and an increased understanding of its tumor microenvironment, efficacious treatment modalities remain elusive.

MICAL2 is a member of the MICAL (molecules interacting with CasL) protein family, evolutionarily conserved flavin monooxygenases whose canonical function is the oxidation and resultant depolymerization of actin. MICAL2 was first linked to malignant disease when its splice variants were found to be overexpressed in prostate cancer, and more recently, studies have revealed that MICAL2 may promote epithelial to mesenchymal transition (EMT), migration and invasion in non-small cell lung cancer (NSCLC), gastric cancer, and breast cancer. However, MICAL2 has not been implicated in pancreatic cancer biology, nor have MICAL2 regulated pathways and the specific roles of MRTF-A versus B in oncogenic phenotypes been comprehensively characterized.

### SUMMARY

Provided herein are methods of treating pancreatic cancer in a subject that include administering to the subject a therapeutically effective amount of a composition comprising an inhibitor of MICAL2, thereby treating the pancreatic cancer in the subject.

In some embodiments, the inhibitor of MICAL2 comprises an inhibitory nucleic acid. In some embodiments, the inhibitory nucleic acid comprises a small interfering RNA (siRNA), a small hairpin RNA (shRNA), an antisense oligonucleotide (ASO), or a small nuclear RNA (snRNA) targeting a MICAL2 nucleic acid. In some embodiments, the inhibitory nucleic acid comprises a small hairpin RNA (shRNA). In some embodiments, the shRNA comprises any one of SEQ ID NOs: 1-3. In some embodiments, the inhibitory nucleic acid comprises a small interfering RNA (siRNA). In some embodiments, the siRNA comprises any one of SEQ ID NOs: 4-11.

Also provided herein are methods of suppressing tumor growth in a subject with cancer that include administering to the subject a therapeutically effective amount of a composition comprising an inhibitor of MICAL2, thereby suppressing tumor growth in the subject.

Also provided herein are methods of suppressing tumor metastasis in a subject with cancer that include administering to the subject a therapeutically effective amount of a composition comprising an inhibitor of MICAL2, thereby suppressing tumor metastasis in the subject.

Also provided herein are methods of enhancing response to chemotherapy in a subject with cancer that include administering to the subject a therapeutically effective amount of a composition comprising an inhibitor of MICAL2, thereby enhancing response to chemotherapy in the subject.

In some embodiments, the cancer is a solid tumor. In some embodiments, the cancer is an appendix cancer, bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer. In some embodiments, the cancer is a pancreatic cancer.

In some embodiments, the inhibitor of MICAL2 comprises an inhibitory nucleic acid. In some embodiments, the inhibitory nucleic acid comprises a small interfering RNA (siRNA), a small hairpin RNA (shRNA), an antisense oligonucleotide (ASO), or a small nuclear RNA (snRNA) targeting a MICAL2 nucleic acid. In some embodiments, the inhibitory nucleic acid comprises a small hairpin RNA (shRNA). In some embodiments, the shRNA comprises any one of SEQ ID NOs: 1-3. In some embodiments, the inhibitory nucleic acid comprises a small interfering RNA (siRNA). In some embodiments, the siRNA comprises any one of SEQ ID NOs: 4-11.

In some embodiments, the method further comprises administering an anti-cancer treatment. In some embodiments, the anti-cancer treatment comprises surgery, administering ionizing radiation, a chemotherapeutic agent, a therapeutic antibody, a checkpoint inhibitor, or any combination thereof. In some embodiments, the anti-cancer treatment comprises a chemotherapeutic agent. In some embodiments, the chemotherapeutic agent comprises vincristine, prednisone, dexamethasone, busulfan, cisplatin, carboplatin, paclitaxel, docetaxel, nab-paclitaxel, altretamine, capecitabine, cyclophosphamide, etoposide (vp-16), gemcitabine, ifosfamide, irinotecan (SN-38), liposomal doxorubicin, melphalan, pemetrexed, topotecan, vinorelbine, goserelin, leuprolide, tamoxifen, letrozole, anastrozole, exemestane, bevacizumab, olaparib, rucaparib, niraparib, nivolumab, pembrolizumab, durvalumab, atezolizumab, radioisotopes, monomethyl auristatin E (MMAE), calicheamicins, deruxtecan, DM1, 5FU, oxaliplatin, and any combinations thereof. In some embodiments, the chemotherapeutic agent comprises gemcitabine.

In some embodiments, the administering comprises intravenous administration, subcutaneous administration, intraperitoneal administration, rectal administration, oral administration, or combinations thereof.

Also provided herein are methods of modulating MICAL2 expression that include delivering an inhibitory nucleic acid into a cell, wherein the inhibitory nucleic acid comprises any one of SEQ ID NOs: 1-11. In some embodiments, the inhibitory nucleic acid inhibits MICAL2 by knockdown of the MICAL2 gene expression.

Also provided herein are compositions comprising an inhibitory nucleic acid of MICAL2, wherein the inhibitory nucleic acid comprises any one of SEQ ID NOs: 1-11.

In some embodiments, the composition is used in a method of treating pancreatic cancer in a subject, and wherein the composition is administered with an anti-cancer treatment. In some embodiments, the anti-cancer treatment comprises surgery, administering ionizing radiation, a chemotherapeutic agent, a therapeutic antibody, a checkpoint inhibitor, or any combination thereof. In some embodiments, the anti-cancer treatment comprises a chemotherapeutic agent. In some embodiments, the chemotherapeutic agent comprises vincristine, prednisone, dexamethasone, busulfan, cisplatin, carboplatin, paclitaxel, docetaxel, nab-paclitaxel, altretamine, capecitabine, cyclophosphamide, etoposide (vp-16), gemcitabine, ifosfamide, irinotecan (SN-38), liposomal doxorubicin, melphalan, pemetrexed, topotecan, vinorelbine, goserelin, leuprolide, tamoxifen, letrozole, anastrozole, exemestane, bevacizumab, olaparib, rucaparib, niraparib, nivolumab, pembrolizumab, durvalumab, atezolizumab, radioisotopes, monomethyl auristatin E (MMAE), calicheamicins, deruxtecan, DM1, 5FU, oxaliplatin, and any combinations thereof. In some embodiments, the chemotherapeutic agent comprises gemcitabine. In some embodiments, the administering comprises intravenous administration, subcutaneous administration, intraperitoneal administration, rectal administration, oral administration, or combinations thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1A shows differentially expressed SE-associated genes in tumor compared to normal tissue. MICAL2 gene is annotated.

FIG. 1B depicts H3K27ac ChIP-seq occupancy upstream and within the MICAL2 loci in aggregated normal and tumor samples. Star denotes the MICAL2 gene start site.

FIG. 1C demonstrates qPCR of MICAL2 using RNA extracted from the same patient samples used for ChIP-seq.

FIG. 1D shows IHC for MICAL2 in normal pancreas and PDAC human tissues.

FIG. 1E shows IHC for MICAL2 in normal pancreas and KPC-derived PDAC mouse tissues.

FIGS. 1F-1G depict survival analysis of PDAC patients segregated by MICAL2 expression (High vs Low) in two datasets: PanCuRx (FIG. 1F) and COMPASS (FIG. 1G).

FIG. 1H shows MICAL2 expression among TCGA cancers.

FIG. 1I shows MICAL2 expression being enriched in PDAC primary tumors and metastases compared to normal pancreatic tissues.

FIG. 2A shows expression of common SRF target genes by qPCR in AsPC1 cells when MICAL2 is knocked down (KD) using siRNA.

FIG. 2B shows many of MRTF/SRF target genes were downregulated as expected.

FIG. 2C shows expression of common SRF target genes by qPCR in KPC46 cells when MICAL2 is knocked down (KD) using siRNA.

FIG. 2D. shows SRF signaling being downregulated in the KPC46 MICAL2 KD cells using an SRF reporter assay.

FIG. 2E show H&E stain of mouse PDAC organoids derived from control KPC46 cells (SCR) and MICAL2-KD (MICAL2).

FIGS. 3A-3B depict immunoblot analyses of AsPC1 (FIG. 3A) and KPC46 (FIG. 3B) cells treated with SCR, MICAL2, MRTF-A and MRTF-B siRNAs at 72 hrs.

FIG. 3C shows immunoblot analysis of BxPc3 cells expressing empty vector (EV) or MICAL2-overexpression vector (OE) at 72 hrs.

FIG. 3D is a representative immunofluorescence picture of AsPC1 cells transfected with siRNA control (SCR) or MICAL2. DAPI marks cell nuclei, and FITC-conjugated dextran are used to label macropinosomes.

FIG. 3E shows quantitation of the relative macropinosomes index from FIG. 3D.

FIGS. 3F-3G show human and mouse models silencing MICAL2, MRTF-A and MRTF-B.

FIG. 3H shows MICAL2 overexpressed (OE) in BxPc3 human PDAC cells.

FIGS. 4A-B show quantification of wound healing assay of AsPC1 (FIG. 4A), and KPC46 (FIG. 4B) transfected with SCR, MICAL2, MRTF-A and MRTF-B siRNAs at the time points indicated.

FIG. 4C represents quantification of wound healing assay of BxPc3 cells expressing empty vector (EV) or MICAL2-overexpression vector (OE) at the time points indicated.

FIGS. 4D-4E show proliferation assays of AsPC1 (FIG. 4D), and KPC46 (FIG. 4E) transfected with SCR, MICAL2, MRTF-A and MRTF-B siRNAs at the time points indicated.

FIG. 4F shows a proliferation assay of BxPc3 cells expressing empty vector (EV) or MICAL2-overexpression vector (OE) at the time points indicated.

FIGS. 4G-4I depicts cell cycle analysis of AsPC1 (FIG. 4G), KPC46 (FIG. 4H) transfected with SCR, MICAL2, MRTF-A and MRTF-B siRNAs at 72 hrs, and BxPc3 (FIG. 4I) cells expressing empty vector (EV) or MICAL2-overexpression vector (OE).

FIG. 5A depicts representative images of subcutaneous AsPC1 tumors grown in immunocompromised mice. AsPC1 cells express shRNA vectors to silence MICAL2, MRTF-A and MRTF-B.

FIG. 5B shows weight quantification of the AsPC1 tumors shown in FIG. 5A.

FIG. 5C shows representative images of subcutaneous KPC46 tumors grown in syngeneic mice. KPC46 cells express shRNA vectors to silence MICAL2, MRTF-A and MRTF-B.

FIG. 5D demonstrates weight quantification of the KPC46 tumors shown in FIG. 5C.

FIG. 5E depicts representative images of subcutaneous BxPc3 tumors grown in immunocompromised mice. BxPc3 cells express EV or MICAL2-OE vectors.

FIG. 5F demonstrates weight quantification of BxPc3 tumors shown in FIG. 5E.

FIG. 6A shows representative images of orthotopic AsPC1 tumors grown in immunocompromised mice. AsPC1 cells express shRNA vectors to silence MICAL2, MRTF-A and MRTF-B.

FIG. 6B depicts weight quantification of the AsPC1 tumors shown in FIG. 6A.

FIG. 6C shows representative images of orthotopic KPC46 tumors grown in syngeneic mice. KPC46 cells express shRNA vectors to silence MICAL2, MRTF-A and MRTF-B.

FIG. 6D shows weight quantification of the KPC46 tumors shown in FIG. 6C.

FIG. 7A demonstrates representative images of liver metastatic burden after splenic injection of KPC46 cell into syngeneic mice. KPC46 cells express shRNA vectors to silence MICAL2, MRTF-A.

FIG. 7B demonstrates representative images of liver metastatic burden after splenic injection of KPC46 cell into syngeneic mice. KPC46 cells express shRNA vectors to silence MICAL2, MRTF-B.

FIG. 7C shows representative images of liver metastatic burden after splenic injection of BxPc3 cells expressing EV or MICAL2-OE vectors into immunocompromised mice.

### DETAILED DESCRIPTION

Pancreatic ductal adenocarcinoma (PDAC) remains a devastating disease that on average, claims the life of an American every 12 minutes. While there has been considerable progress in understanding numerous aspects of PDAC biology, this has not yet translated to significant progress in the clinic. Resistance to current cytotoxic therapies remains nearly universal and thus novel therapeutic strategies are desperately needed to improve patient outcome.

Regulatory regions of the genome, termed “super enhancers”, are responsible for the transcription of genes defining cell identity and may represent novel therapeutic targets for PDAC. Previous studies have indicated that histone three lysine-27 acetylation (H3K27ac) marks serve as a reliable indicator for demarcating super-enhancers efficiently and robustly, wherein these chromatin regions can regulate key genes that govern cell phenotype. In tumor cells, this regulatory mechanism may encompass both oncogene and non-oncogene drivers of the transformed state. It has been known that pancreatic ductal adenocarcinoma (PDAC) may be driven and sustained by the activation of super-enhancers and that delineating the genes associated with these super-enhancers could unveil therapeutic targets for drug development. Using chromatin immunoprecipitation and sequencing (e.g., ChIPSeq), MICAL2 was identified as a super enhancer associated gene in human PDAC samples and its overexpression at the RNA and protein level was confirmed in both human tissues, cell lines as well in murine models.

MICAL2 is a member of the MICAL (molecules interacting with CasL) protein family, evolutionarily conserved flavin monooxygenases whose canonical function is the oxidation and resultant depolymerization of actin. Unique to its other family members MICAL1 and 3, MICAL2 has no autoinhibitory domain and is thus, constitutively active. MICAL2, which is expressed in both the cytoplasm and the nucleus, was previously shown to indirectly regulate serum response factor (SRF) mediated transcription through its modulation of nuclear G actin levels, wherein G actin acts to sequester myocardin related transcription factors (MRTF), coactivators of SRF. Nuclear accumulation of MRTFs is associated with upregulation of genes associated with cell migration, fibrosis, and epithelial to mesenchymal transition (EMT), although MRTF-A has been the subject of most cancer related studies. While the role of MRTF-B in oncogenesis is much more uncertain, with some studies linking it to mesenchymal and hepatocellular tumor progression, a recent study concluded it acts as tumor suppressor in human and murine colorectal cancer. MICAL2 was first linked to malignant disease when its splice variants were found to be overexpressed in prostate cancer, and more recently, studies have revealed that MICAL2 may promote EMT, migration and invasion in non-small cell lung cancer (NSCLC), gastric cancer, and breast cancer. However, MICAL2 has not been implicated in pancreatic cancer biology, nor have MICAL2 regulated pathways and the specific roles of MRTF-A versus B in oncogenic phenotypes been comprehensively characterized. As MICAL2 has been identified as a highly ranked super-enhancer-associated gene in human PDAC, in some embodiments, MICAL2 expression can be correlated with poor prognosis in patients who had undergone surgical resection. In some embodiments, MICAL2 promotes PDAC growth and metastasis, and also be associated with patterns of gene expression associated with KRAS. Thus, the present disclosure describes methods of using MICAL2 as a potential therapeutic target for PDAC.

Several methods are described herein, including methods of treating pancreatic cancer in a subject that include administering to the subject a therapeutically effective amount of a composition comprising an inhibitor of MICAL2. In some embodiments, provided herein are methods of suppressing tumor growth and/or tumor metastasis, or enhancing response to chemotherapy in a subject with cancer by administering to the subject a therapeutically effective amount of a composition comprising an inhibitor of MICAL2. Also provided herein are methods of modulating MICAL2 expression by delivering an inhibitory nucleic acid into a cell, wherein the inhibitory nucleic acid comprises a sequence selected from SEQ ID NOs: 1-11. Also described herein are compositions that include an inhibitory nucleic acid of MICAL2, wherein the inhibitory nucleic acid comprises a sequence selected from SEQ ID NOs: 1-11.

Various non-limiting aspects of these methods and compositions are described herein and can be used in any combination without limitation. Additional aspects of various components of the methods and compositions described herein are known in the art.

It must be noted that, as used in the specification and the appended claims, the singular forms “a,” “an” and “the” include plural referents unless the context clearly dictates otherwise.

As used herein, the terms “cancer”, “tumor”, and “carcinoma” refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a metastatic solid tumor. In general, examples of different types of cancers known in the art include, for example, a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer. In some embodiments, hematopoietic cancers can include leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, precancerous pathology such as myelodysplastic syndromes, acquired aplastic anemia, Fanconi anemia, paroxysmal nocturnal hemoglobinuria (PNH) and 5q-syndrome and the like.

As used herein, a “cell” can refer to a eukaryotic cell, optionally obtained from a subject or a commercially available source.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, “nucleic acid” or “nucleic acid molecule” is used to include any compound and/or substance that comprise a polymer of nucleotides. In some embodiments, a polymer of nucleotides is referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” or “nucleic acid molecule” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

As used herein, the term “subject” refers to an organism, typically a mammal (e.g., a human). In some embodiments, a subject is suffering from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, the subject can be an animal, human or non-human. Non-limiting examples of non-human subjects can include mice, rats, hamsters, rabbits, cats, dogs, horses, pigs, donkeys, monkeys, and/or other non-human primates such as apes and lemurs. In some embodiments, the subject is a human.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

### Methods of Treating Pancreatic Cancer

Provided herein are methods of treating pancreatic cancer in a subject that include administering to the subject a therapeutically effective amount of a composition comprising an inhibitor of MICAL2, thereby treating the pancreatic cancer.

As used herein, the term “treating” means a reduction in the number, frequency, severity, or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be oral, enteral, parenteral, etc. In some particular embodiments, administration may be bronchial (e.g., by bronchial instillation), buccal, enteral, intra-arterial, intragastric, intramedullary, intramuscular, intranasal, intraperitoneal, intrathecal, intravenous, intraventricular, intracisternal, within a specific organ (e.g., intrahepatic), mucosal, nasal, oral, rectal, subcutaneous, sublingual, topical, tracheal (e.g., by intratracheal instillation), by patch, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may involve methods of delivery that include, but are not limited to, use of external and/or implanted infusion pumps, liquid formulation, capsulated formulation, or slow-release encapsulation.

As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

### Inhibitor of MICAL2

In some embodiments, an inhibitor of MICAL2 inhibits expression of a MICAL2 gene. In some embodiments, an inhibitor of MICAL2 comprises an inhibitory nucleic acid. Inhibitory nucleic acids in any of the methods and compositions described herein can include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of a target RNA (e.g., MICAL2 gene) and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or any combinations thereof. See, e.g., WO 2010040112, which is herein incorporated by reference in its entirety. In some embodiments, the inhibitory nucleic acid inhibits MICAL2 by knockdown of the MICAL2 gene expression. In some embodiments, an inhibitory nucleic acid comprises a small interfering RNA (siRNA), a small hairpin RNA (shRNA), an antisense oligonucleotide (ASO), or a small nuclear RNA (snRNA) targeting a MICAL2 nucleic acid.

siRNA/shRNA

In some embodiments, an inhibitory nucleic acid can comprise an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering RNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region, and a loop region. Such an RNA molecule when expressed desirably forms a “hairpin” structure and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99 (6), 5515-5520, (2002); Yu et al. Proc NatlAcadSci USA 99:6047-6052, (2002).

In some embodiments, an inhibitory nucleic acid comprises a small hairpin RNA (shRNA). In some embodiments, the shRNA comprises a sequence selected from SEQ ID NOs: 1-3. In some embodiments, the inhibitory nucleic acid has at least 95% sequence identity (e.g., at least 96%, at least 97%, at least 98%, at least 99%) to a sequence selected from shRNA SEQ ID NOs: 1-3.

TABLE 1
Human (h) and Mouse (m) shRNA sequences

| | NUCLEIC ACID | SEQ ID |
|---|---|---|
| | SEQUENCE | NO: |
| h-MICAL2-sh pSMART | TGAGAACTTT | 1 |
| hCMV/TurboGFPMICAL2 | GTCCAGGC | |
| m-MICAL2-sh pSMART | GTGCTGGCTC | 2 |
| hCMV/TurboGFPMICal2 | CATCGACCA | |
| SMARTvector Non- | CACACAACAT | 3 |
| Targeting | GTAAACCAGG | |
| Control Target | GA | |

In some embodiments, an inhibitory nucleic acid comprises a small interfering RNA (siRNA). In some embodiments, the siRNA comprises a sequence selected from SEQ ID NOs: 4-11. In some embodiments, the inhibitory nucleic acid has at least 95% sequence identity (e.g., at least 96%, at least 97%, at least 98%, at least 99%) to a sequence selected from siRNA SEQ ID NOs: 4-11.

TABLE 2
Human (h) and Mouse (m) siRNA sequences

| | NUCLEIC ACID SEQUENCE | SEQ ID NO: |
|---|---|---|
| Mouse | AAGGAGUACAAGCGAGGAA | 4 |
| Mical2 | CCACAUCAGUAUCCGACAA | 5 |
| siRNA | CUAAGGAGAUGGACCGCUU | 6 |
| | AGGGAUUGAUCUCGAGAAC | 7 |
| Human | GGACGAGCCUUCAAACUUU | 8 |
| Mical2 | GCAUCCACGUGCAAAGGUA | 9 |
| siRNA | GCAGGAACGCCGUGUCUCA | 10 |
| | UAUCAGUAUUCGCCAACUA | 11 |

In some embodiments, an inhibitory nucleic acid can be 10 to 50 (e.g., 10 to 40, 10 to 35, 10 to 30, 10 to 20, 20 to 50, 20 to 40, 20 to 30, 30 to 50, 30 to 40, or 40 to 50) nucleotides in length. In some embodiments, an inhibitory nucleic acid can have a complementary portion of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin.

In some embodiments, an inhibitory nucleic acid is sufficiently complementary to the target RNA (e.g., MICAL2 gene), i.e., hybridize sufficiently well and with sufficient specificity, to give the desired effect. As used herein, “complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a target RNA, then the bases are considered to be complementary to each other at that position. In some embodiments, 100% complementarity is not required. In some embodiments, an inhibitory nucleic acid described herein can have at least 80% sequence complementarity to a target region within the target RNA, e.g., 90%, 95%, or 100% sequence complementarity to the target region within the target RNA. The inhibitory nucleic acid can be directed to hybridize sufficiently well (least 80% sequence complementarity) and with sufficient specificity to one or more of the human MICAL2 sequences corresponding to NM_001393937.1; NM_001346293.2; NM_001346297.2; NM_014632.4; NM_001346295.2; NM_001282663.2; NM_001346299.2; NM_001346296.2; NM_001346292.2; NM_001346298.2; NM_001346294.2; NM_001282668.2; NM_001282664.1; NM_001282665.1; NM_001282666.1; or NM_001282667.1.

For further disclosure regarding inhibitory nucleic acids, see, e.g., US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids), which are herein incorporated by reference in their entireties.

### Methods of Suppressing Tumor Growth/Metastasis

Provided herein are methods of suppressing tumor growth and/or metastasis in a subject with cancer that include administering to the subject a therapeutically effective amount of a composition comprising an inhibitor of MICAL2. Also provided herein are methods of enhancing response to chemotherapy in a subject with cancer that include administering to the subject a therapeutically effective amount of a composition comprising an inhibitor of MICAL2.

In some embodiments, a subject is diagnosed as having a cancer. In some embodiments, the cancer is a solid tumor. In some embodiments, the cancer is an appendix cancer, bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer. In some embodiments, the cancer is a pancreatic cancer.

In some embodiments, any one of the methods described herein can further include administering an anti-cancer treatment. In some embodiments, an anti-cancer treatment comprises surgery, administration of ionizing radiation, a chemotherapeutic agent, a therapeutic antibody, a checkpoint inhibitor, or any combination thereof. In some embodiments, an anti-cancer treatment comprises a chemotherapeutic agent.

As used herein, the term “chemotherapeutic agent” can refer to one or more pro-apoptotic, cytostatic and/or cytotoxic agents, for example specifically including agents utilized and/or recommended for use in treating one or more diseases, disorders or conditions associated with undesirable cell proliferation. In some embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some embodiments, a chemotherapeutic agent may be utilized in the context of an antibody-drug conjugate.

In some embodiments, a chemotherapeutic agent comprises vincristine, prednisone, dexamethasone, busulfan, cisplatin, carboplatin, paclitaxel, docetaxel, nab-paclitaxel, altretamine, capecitabine, cyclophosphamide, etoposide (vp-16), gemcitabine, ifosfamide, irinotecan (SN-38), liposomal doxorubicin, melphalan, pemetrexed, topotecan, vinorelbine, goserelin, leuprolide, tamoxifen, letrozole, anastrozole, exemestane, bevacizumab, olaparib, rucaparib, niraparib, nivolumab, pembrolizumab, durvalumab, atezolizumab, radioisotopes, monomethyl auristatin E (MMAE), calicheamicins, deruxtecan, DM1, 5FU, oxaliplatin, and any combinations thereof. In some embodiments, the chemotherapeutic agent comprises gemcitabine.

### Methods of Modulating MICAL2 Expression

Also provided herein are methods of modulating MICAL2 expression that include delivering an inhibitory nucleic acid into a cell, wherein the inhibitory nucleic acid comprises a small hairpin RNA (shRNA) comprising a sequence selected from SEQ ID NOs: 1-3, or a small interfering RNA (siRNA) comprising a sequence selected from SEQ ID NOs: 4-11.

As used herein, “modulating” can refer to modifying, regulating, or altering the endogenous gene expression in a cell. In some embodiments, modulating gene expression can include systematically influencing RNA stability and/or translation by activating or suppressing the gene expression. In some embodiments, modulation of gene expression can include stabilizing a target RNA. In some embodiments, stabilizing a target RNA can increase translation of the target RNA. In some embodiments, modulation of gene expression can include destabilizing a target RNA. In some embodiments, destabilizing a target RNA can suppress translation of the target RNA. In some embodiments, modulation of gene expression can include increasing translation of a target RNA. In some embodiments, modulation of gene expression can include suppressing translation of a target RNA. In some embodiments, the gene expression of the target RNA is upregulated. In some embodiments, the gene expression of the target RNA is downregulated. In some embodiments, the inhibitory nucleic acid inhibits MICAL2 by knockdown of the MICAL2 gene expression.

### Pharmaceutical Compositions

The methods described herein can include the administration of pharmaceutical compositions and formulations comprising an inhibitor of MICAL2. In some embodiments, an inhibitor of MICAL2 comprises an inhibitory nucleic acid. Also described herein are compositions that include an inhibitory nucleic acid of MICAL2, wherein the inhibitory nucleic acid comprises a sequence selected from SEQ ID NOs: 1-11. In some embodiments, a composition is used in a method of treating cancer, e.g., pancreatic cancer, in a subject, and the composition is administered with an anti-cancer treatment.

In some embodiments, the pharmaceutical compositions are formulated with a pharmaceutically acceptable carrier. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

In some embodiments, any one of the inhibitory nucleic acids described herein can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the pharmaceutical compositions described herein include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences as described herein) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical as described herein and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., US20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations can be administered for prophylactic and/or therapeutic treatments. In some embodiments, for therapeutic applications, compositions are administered to a subject who is at risk of or has a disorder described herein, in an amount sufficient to cure, alleviate or partially arrest the clinical manifestations of the disorder or its complications; this can be called a therapeutically effective amount.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age, and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as described herein are correct and appropriate.

Single or multiple administrations of formulations can be given depending on for example: the dosage and frequency as required and tolerated by the patient, the degree and amount of therapeutic effect generated after each administration, and the like. The formulations should provide a sufficient quantity of active agent to effectively treat, prevent or ameliorate conditions, diseases or symptoms.

### EXAMPLES

The disclosure is further described in the following examples, which do not limit the scope of the disclosure.

### Example 1—MICAL2 is a Super Enhancer Associated Gene in Human PDAC

Tumor-specific active super enhancer (SE) regions were identified in human PDAC. Tumors were obtained (n=7) from surgical resections of PDAC and normal pancreatic tissues (n=6) from patients undergoing resection for non-adenocarcinoma histology. Tissues were lysed and histone 3 lysine 27 acetylation (H3K27ac) ChIP-seq was performed to identify regions of active transcription in human PDAC tissues. SE regions containing SE-associated genes were identified computationally for both normal and tumor sample. Hierarchical clustering of the SE-associated genes was used to define those that were enriched in tumor or normal tissues. Gene set enrichment analysis (GSEA) of SE-associated genes was utilized to determine which pathways were enriched in tumor versus normal-adjacent tissues. Overall, it was found that immune-related genes were upregulated in tumor-associated genes, while pancreatic endocrine genes were correlated with normal tissues. Among the tumor-specific SE-associated genes LIF was found which was previously identified as a potential therapeutic target in PDAC and is a target of ongoing clinical trials.

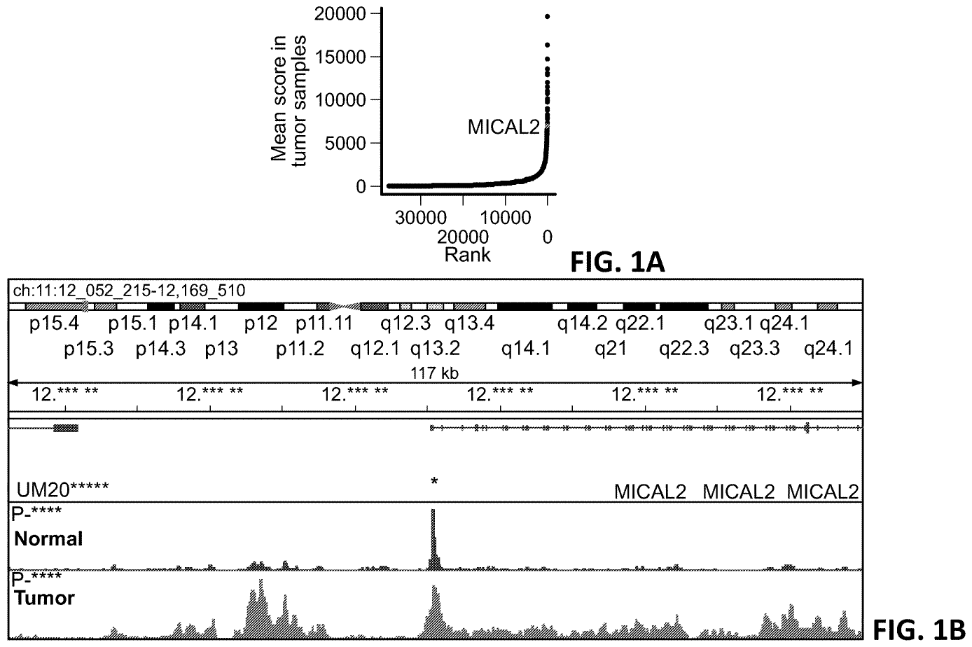

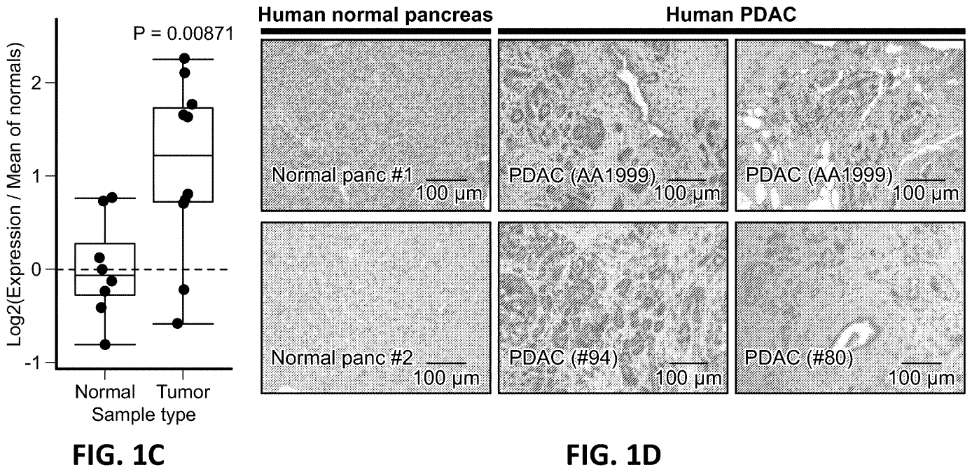

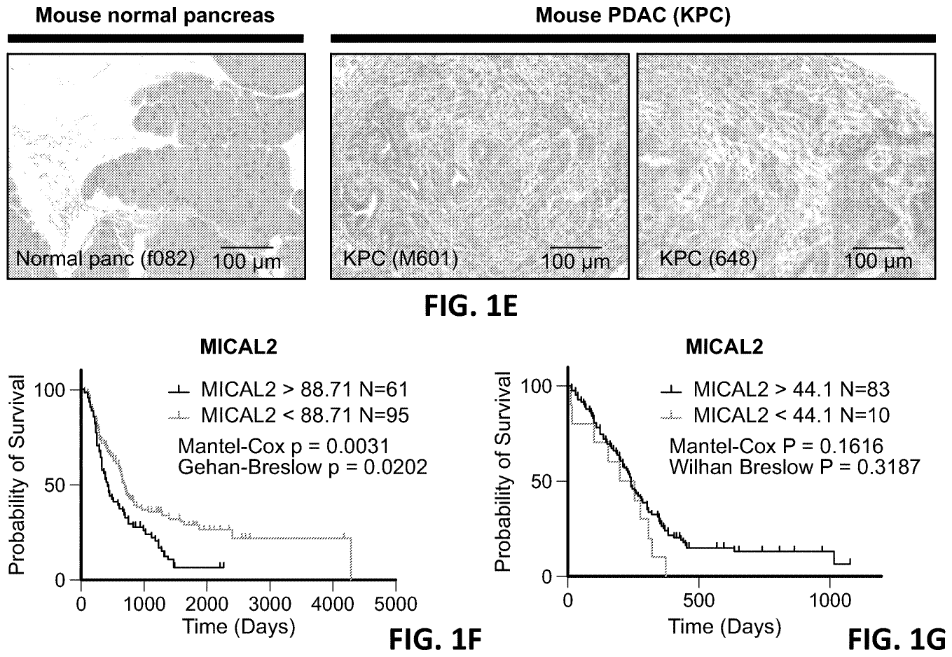

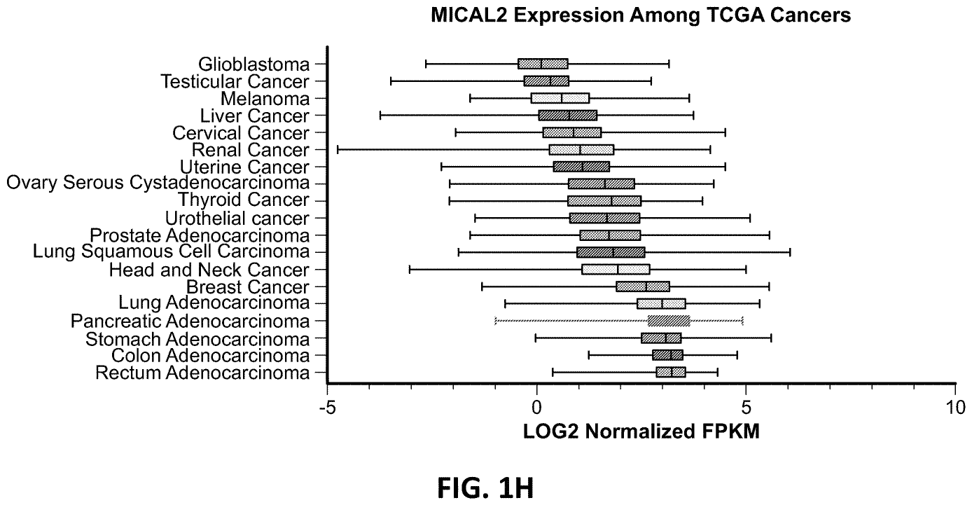

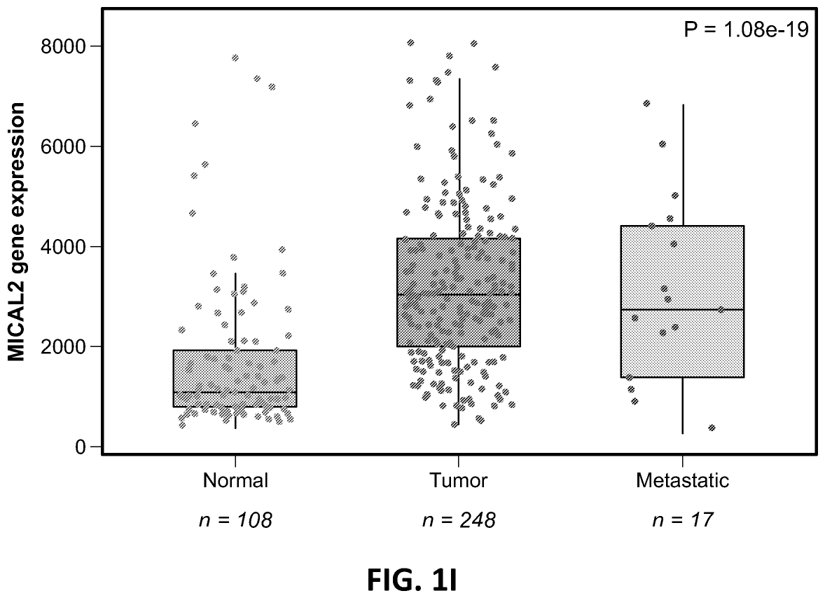

It was next sought to identify SE-associated genes that were upregulated in PDAC, and which encoded proteins with the potential to be targeted by small molecules or antibody-based therapeutics. One of the top hits in the analysis was MICAL2, a member of the Molecule Interacting with CasL (MICAL) family. MICAL2 contains several functional protein domains including a flavin monooxygenase domain. MICAL2 was significantly enriched in tumor samples compared to normal (FIGS. 1A-1B). Furthermore, quantitative PCR analysis revealed that MICAL2 mRNA was enriched in tumor samples suggesting that the increased H3K27ac at the promoter and coding region does lead to increased transcriptional activation of the MICAL2 loci (FIG. 1C). Investigation of TCGA solid cancers datasets revealed that PDAC is the 4th highest expressor of MICAL2 (FIG. 1H). Furthermore, using the TNMplot dataset, it was determined that MICAL2 expression was significantly enriched in PDAC primary tumors and metastases compared to normal pancreatic tissues (FIG. 1I). To ascertain if the higher level of MICAL2 transcription resulted in greater MICAL2 protein levels within PDAC tumors, IHC was performed on human normal pancreas and PDAC tissues. A tumor-specific increase was found in MICAL2 staining across 4 patients' tumors (FIG. 1D). It was confirmed that high MICAL2 protein levels and tumor-specificity is preserved in the commonly used Kras^{LSL-G12D}; TP53^{LSL-R172H}, PDX1-cre (KPC) genetically engineered mouse model (FIG. 1E). In addition, it was found that MICAL2 was significantly overexpressed in mouse KC and KPC organoids. The expression of MICAL2 across commonly used PDAC cell lines was also investigated and it was found that the majority of lines express MICAL2; ASPC1 is a high MICAL2 expressor while BxPc3 and MIAPaCa2 express low to no MICAL2.

Finally, the association of MICAL2 expression with outcomes in PDAC patients that were eligible for surgical intervention (PanCuRx) and in patients with advanced disease (COMPAS) was investigated. It was found that high MICAL2 expression correlated with worse outcome in the surgical cohort but was not prognostic in the advanced cohort (FIGS. 1F-1G). This suggested a possible role for MICAL2 in progression from primary to advanced disease. Overall, it was found that PDAC has a distinct landscape of SE-associated genes that are linked with known PDAC biology. MICAL2 was found among these genes and it was determined that MICAL2 transcription is higher in PDAC compared to normal adjacent tissues both in this study as well as in other independent datasets and that this is associated with increased expression at the protein level. Importantly MICAL2 is associated with a poorer prognosis in patients whose tumor were surgically removed indicating expressing high MICAL2 may mark tumors at increased for recurrence and progression.

### Example 2—MICAL2 Expression is Associated with KRAS and EMT Signaling Pathways

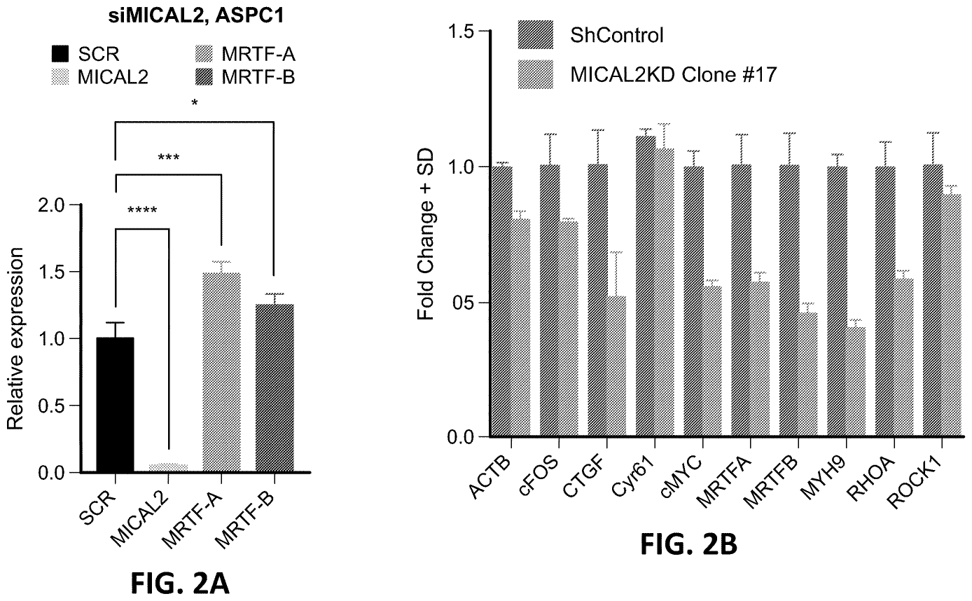

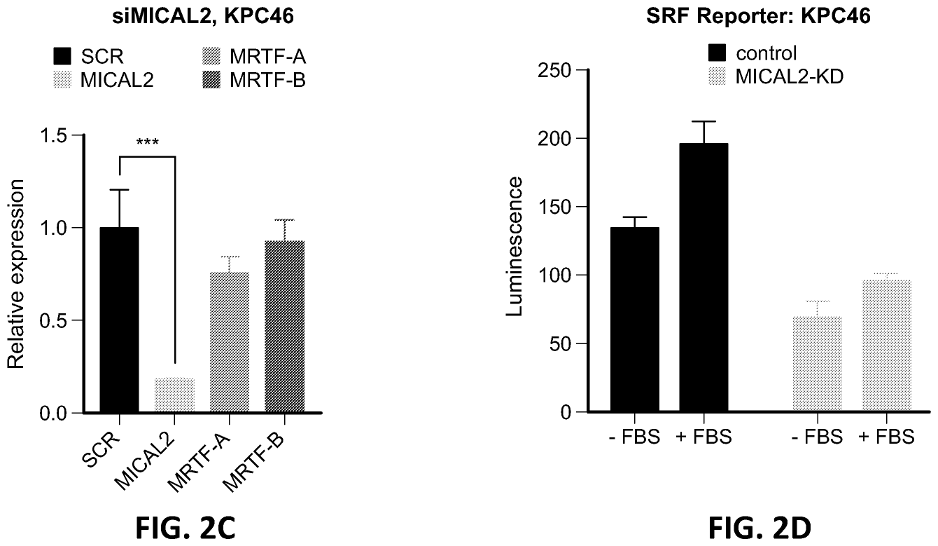

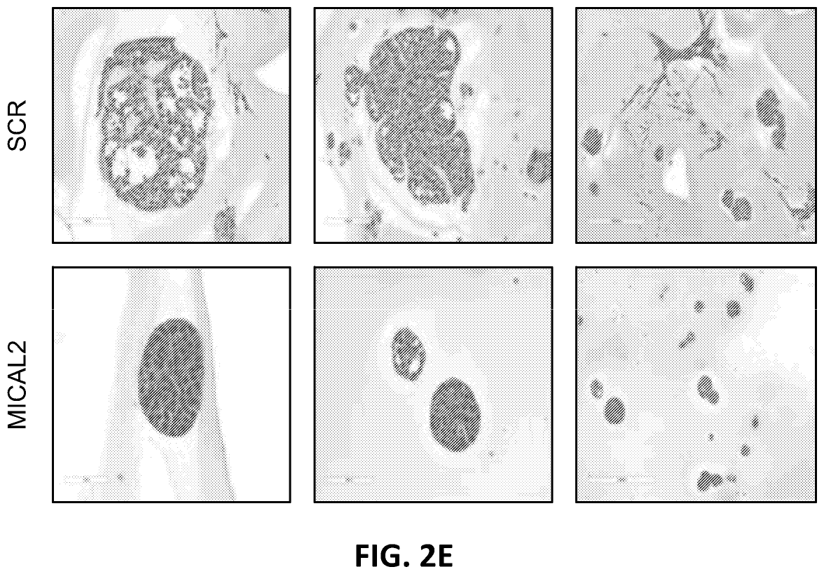

As MICAL2 is known to canonically regulate MRTF/SRF activity, it was first sought to determine if this was occurring in the setting of PDAC. The expression of common SRF target genes was checked by qPCR in ASPC1 cells when MICAL2 is knocked down (KD) using siRNA (FIG. 2A). Interestingly, in ASPC1 cells, the KD of MICAL2 led to the significant over expression and possible compensation of MRTF-A and-B. It was also found that many of MRTF/SRF target genes were downregulated as expected (FIG. 2B). RhoA expression was dramatically reduced in MICAL2 KD cells, suggesting that MICAL2 may regulate a key promoter and target of SRF signaling. To further understand the impact of MICAL2 on pancreatic cancer cell biology RNA sequencing was performed. Using differential gene expression analysis comparing siRNA targeting MICAL2 and scramble control, it was found as expected that MICAL2 was the most significantly repressed gene. To investigate pathways likely to be regulated by MICAL2, gene set enrichment analysis (GSEA) was performed. Interestingly, it was found that KRAS signaling pathways were dramatically reduced in ASPC1 cells lacking MICAL2. Additional pro-survival pathways were lost in the MICAL2 KD cells such as TNFa and HIF1a signaling suggesting that MICAL2 may act as a proto-oncogene in PDAC. Importantly, it was found that epithelial to mesenchymal transition (EMT) signaling was also significantly reduced in MICAL2 KD cells. As an orthogonal pathway analysis method, OncoGPS methodology was used to define cell states associated with MICAL2 expression in other tumor models of the Cancer Cell Line Encyclopedia (CCLE). Similar to GSEA, it was found that the EMT state was associated with higher MICAL2 gene expression in the CCLE. To further investigate the EMT phenotype, a murine PDAC organoid line was used derived from a KPC liver metastasis, KPC46, which has mesenchymal features and is highly metastatic (FIG. 2C). After MICAL2 sh-based knockdown, mesenchymal to epithelial changes were observed, marked by highly uniform, polarized, compact epithelial cell structures (FIG. 2E). It was further validated that SRF signaling was downregulated in the KPC46 MICAL2 KD cells using an SRF reporter assay (FIG. 2D). Overall, these experiments revealed that MICAL2 drives MRTF/SRF transcription, EMT and pro-oncogenic pathways in both human and mouse models of PDAC.

### Example 3—MICAL2 Promotes KRAS Signaling

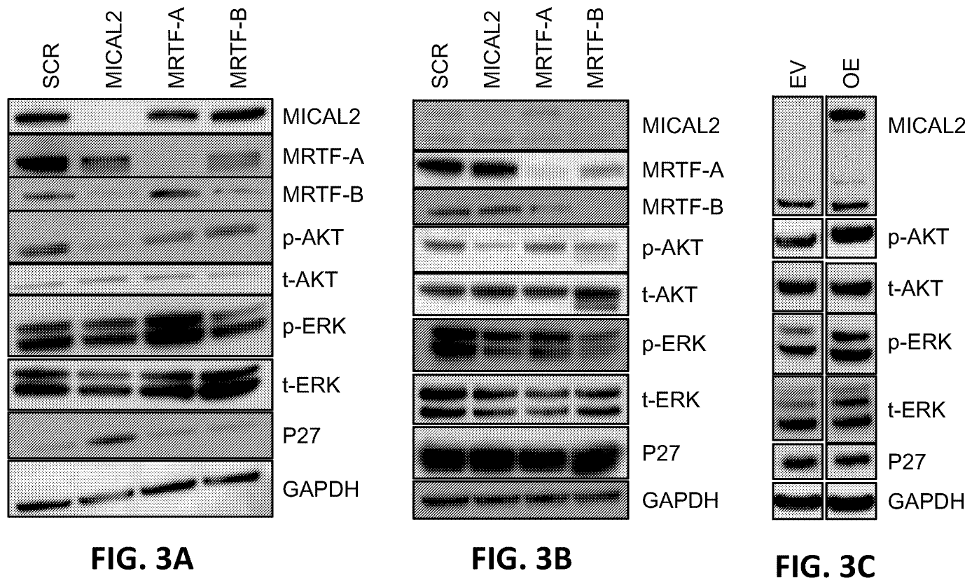

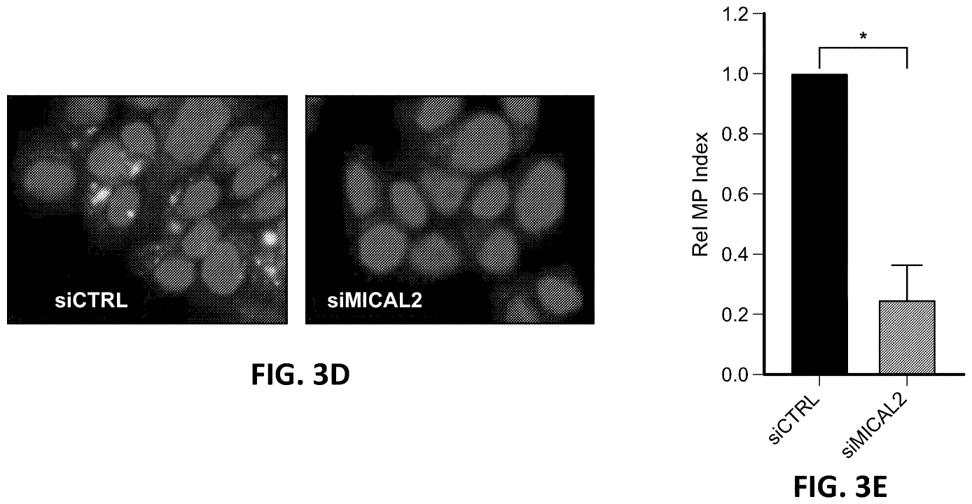

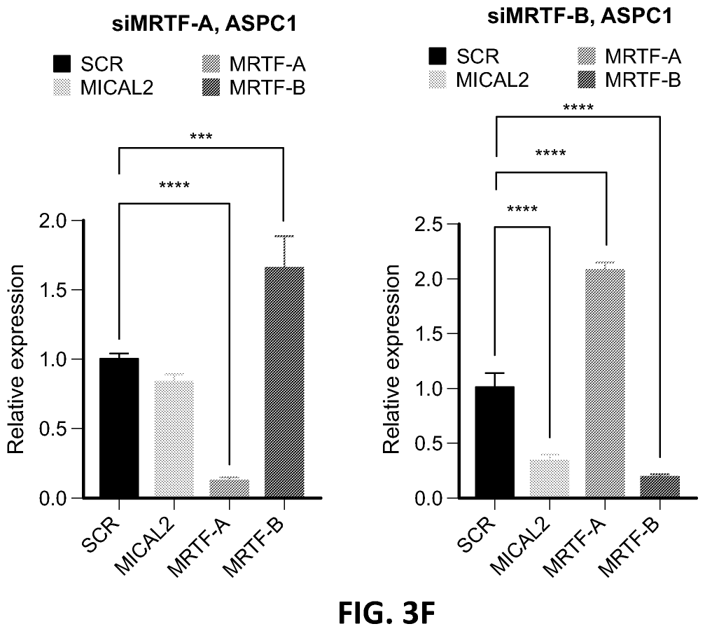

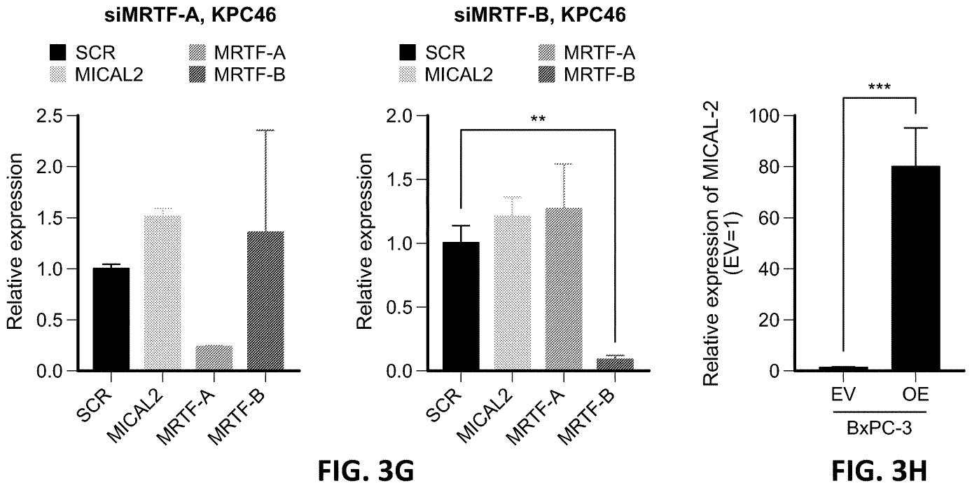

The transcriptomic analyses indicated that KRAS signaling is altered in MICAL2 deficient PDAC cells. Therefore, activation of PI3K and MAPK signaling cascade was evaluated in cells with loss and gain of function of MICAL2. Since MICAL2 loss leads to altered expression of the SRF co-activators MRTF-A and MRTF-B, the effect of KD of these two genes were also investigated. Human and mouse models silencing MICAL2, MRTF-A and MRTF-B were generated (FIGS. 3F-3G). For gain of function studies, MICAL2 was overexpressed (OE) in BxPc3 human PDAC cells as BxPc3 does not endogenously express MICAL2 (FIG. 3H). In the human PDAC cell line, ASPC1, loss of MICAL2 led to a marked decrease in p-AKT, and a minor decrease in p-ERK1/2 (FIG. 3A). The negative cell cycle regulator P27 was dramatically increased in MICAL2 KD cells suggesting a possible mechanism for decreased cell proliferation. The siRNA mediated silencing of MRTF-B phenocopied the p-AKT and p-ERK1/2 decrease observed in MICAL2 silenced cells. Notably, MICAL2 KD led to a partial loss of MRTF-B protein while the RNA was not decreased (FIG. 2A). Interestingly, the loss of MRTF-A did not recapitulate this phenotype and neither MRTFs' loss of function phenocopied the P27 increase. Next, the loss of MICAL2 and MRTFs was evaluated in KPC46 cells. It was found that mouse PDAC cells recapitulated the decrease of p-AKT and pERK1/2 in both MICAL2 and MRTF-B silenced cells, however, P27 was unchanged in this cell line (FIG. 3B). When BxPc3 cells modified to express MICAL2 were examined, it was found that p-AKT, p-ERK, and P27 expression were increased (FIG. 3C). These results demonstrate that MICAL2 and MRTF-B deficient cells have reduced phosphorylation of PI3K and MAPK consistent with reduced KRAS signaling in these cells. To further assess the impact of MICAL2 on KRAS signaling, the effects of MICAL2 KD on macropinocytosis was examined, an extracellular nutrient scavenging process driven by KRAS. A dramatic reduction was observed in macropinocytosis in MICAL2 deficient cells which is also consistent with a decrease in KRAS signaling activity (FIGS. 3D-3E). In sum, loss and gain of function studies revealed that MICAL2 promotes signaling through KRAS as manifest by phosphorylation of MAPK and PI3K and macropinocytosis. These observed biochemical results were thus consistent with the pathway analyses of the cellular transcriptome.

### Example 4—MICAL2 Promotes PDAC Cell Proliferation and Migration

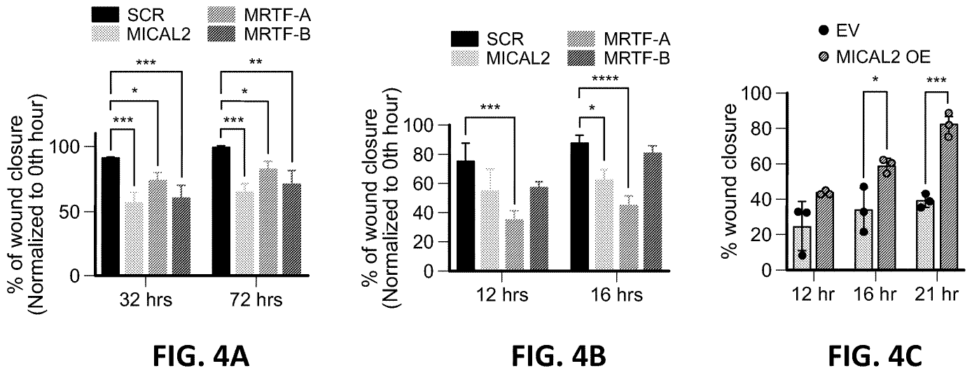

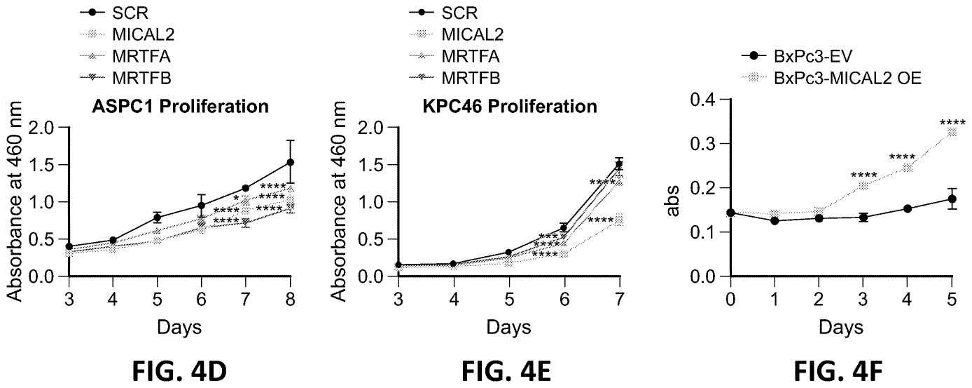

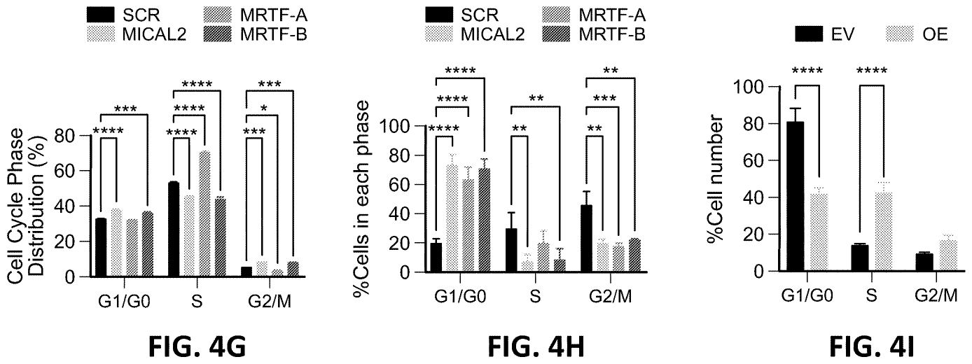

It was next sought to investigate how MICAL2 loss and gain of function would impact oncogenic phenotypes in PDAC cells. Since MICAL2 appeared to drive EMT, the motility of MICAL2, MRTF-A and MRTF-B deficient cells was first measured. It was found that migration in both ASPC1 and KPC46 was reduced after MICAL2 KD (FIGS. 4A-4B). It was also found that MRTF-A and -B KD decreased migration in the human ASPC1 cells, while in mouse KPC46 only MRTF-A KD resulted in reduced migration. Conversely, in a gain of function experiment, BxPc3 cells overexpressing MICAL2 had increased migration than empty vector control cells (FIG. 4C). Using an invasion assay, it was further determined that KPC46 cells with loss of MICAL2 were less capable of invading into a gelatin matrix.

Next, the impact of MICAL2 on cell proliferation was assessed. It was found that MICAL2 loss led to a significant decrease in proliferation in ASPC1 and KPC46 cells, while OE of MICAL2 in BxPc3 led to an increase in proliferation rate compared to control cells (FIGS. 4D-4F). Interestingly, KD of either MRTFs in the human or mouse PDAC cells led to only a partial decrease in the cell proliferation rate (FIGS. 4D-4E). To better understand which part of the cell cycle was impacted by MICAL2 expression, flow cytometry was used to examine cell cycle progression. In both ASPC1 and KPC46, MICAL2 and MRTF-B deficient cells had a significant shift toward arrest in G0/G1 phase and concomitant reduction in the proportion of cells in S phase and G2/M (FIGS. 4G-4H). MRTF-A depleted KPC46 cells had a cell cycle profile but ASPC1 cells lacking MRTF-A surprisingly had a block in S phase rather than G0/G1 hinting at differences between models and possibly between human and mouse PDAC cells. Conversely, BxPc3 MICAL2-OE cells progressed faster through G0/G1 and had an increased S phase proportion compared to control indicating that the increase MICAL2 expression was sufficient to increase cell division (FIG. 4I).

Overall, these experiments show that MICAL2 and MRTF-A/B expression promote cell migration invasion, and proliferation. Further, these findings are consistent with the RNAseq and biochemical results we observed after genomic knockdown of MICAL2 in PDAC cells.

### Example 5—MICAL2 and MRTF-B Promote Heterotopic and Orthotopic Growth In Vivo

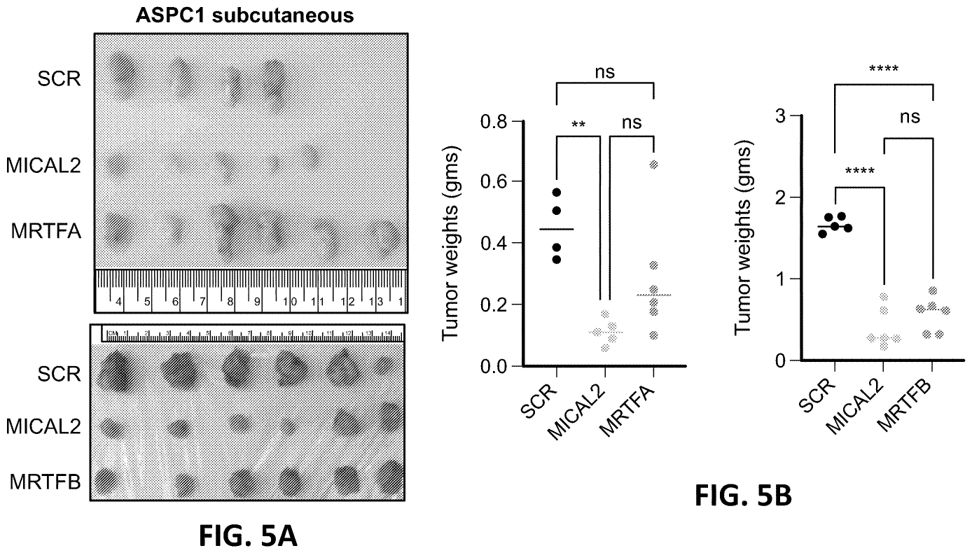

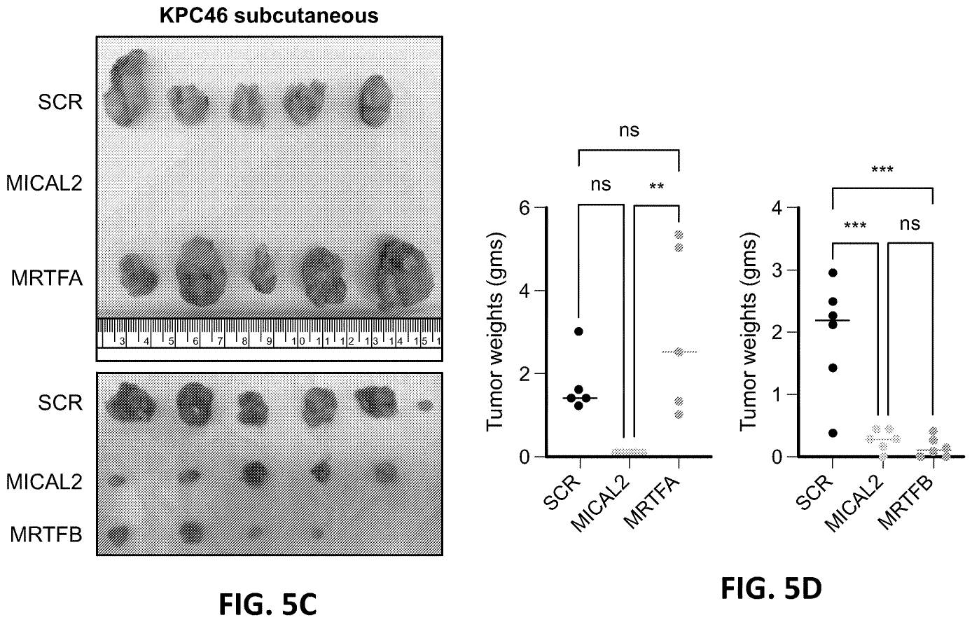

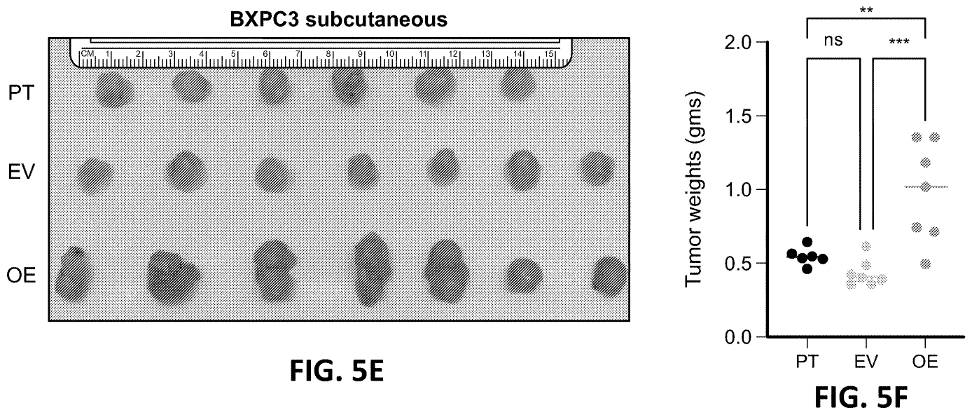

When MICAL2 was silenced in vitro, reduced cell proliferation, migration, invasion and a reversal of the EMT phenotype were observed. Therefore, it was next sought to determine how loss of MICAL2 and MRTFs impacted tumorigenesis initially using heterotopic subcutaneous mouse transplant models. First, ASPC1 cells with constitutive KD of MICAL2 or MRTF-A/B were transplanted into immunodeficient NSG mice. A dramatic decrease in tumor size was observed when MICAL2 and MRTF-B were lost but no significant differences after MRTF-A silencing (FIGS. 5A-5B). Transplantation of KPC46 cells in syngeneic mice recapitulated the results observed with human PDAC cells, though with an even more profound reduction in tumor formation (FIGS. 5C-5D). In one experiment, no tumors formed from the MICAL KD cells while in a repeat experiment 1/6 tumors didn't form, and the remaining tumors were markedly growth inhibited relative to control. Conversely, BxPc3 MICAL2-OE cells grew larger than control cells when injected into the flank of NSG animals (FIGS. 5E-5F).

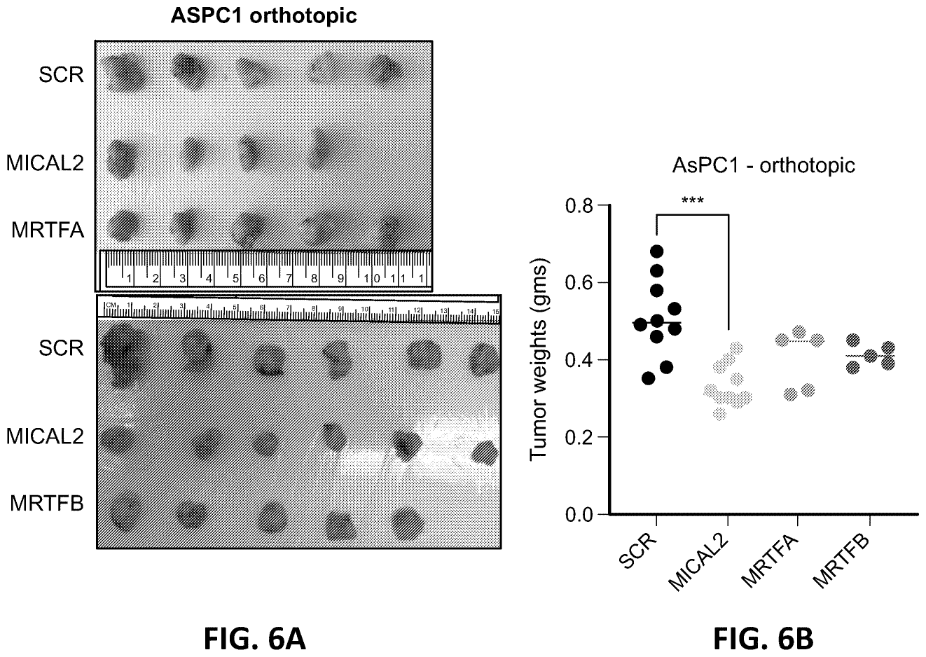

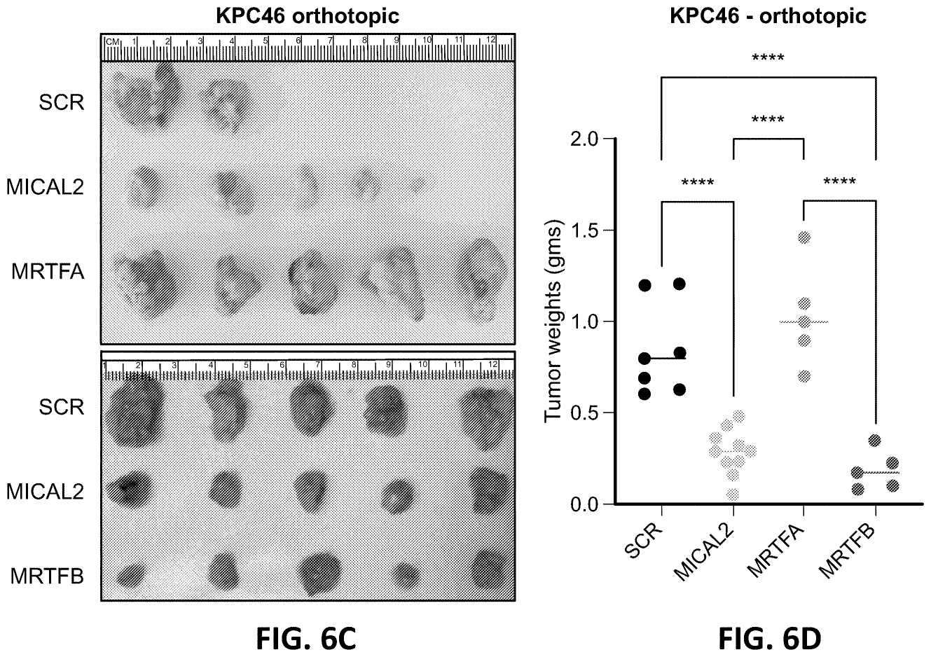

It was next sought to investigate how MICAL2 and MRTFs impact orthotopic tumor growth by implanting cells into the pancreatic tail. It was found that only the mice implanted with ASPC1 MICAL2-KD cells, not the MRTF-KDs had a significantly decreased tumor burden, whereas in the KPC46 model, both the MICAL2 and MRTF-B silenced cells had reduced in vivo growth compared to scramble control (FIGS. 6A-6D). MRTF-A again had no impact on tumor growth. In summary, expression of MICAL2 and MRTF-B in PDAC cells is critical for tumorigenesis in both heterotopic and orthotopic locations whereas MRTF-A expression did not impact tumor growth. These results are consistent with the in vitro findings that MICAL2 promotes PDAC cell proliferation and suggest important functional consequences related to MRTF isoform expression.

### Example 6—MICAL2 Promotes Metastasis in Mice

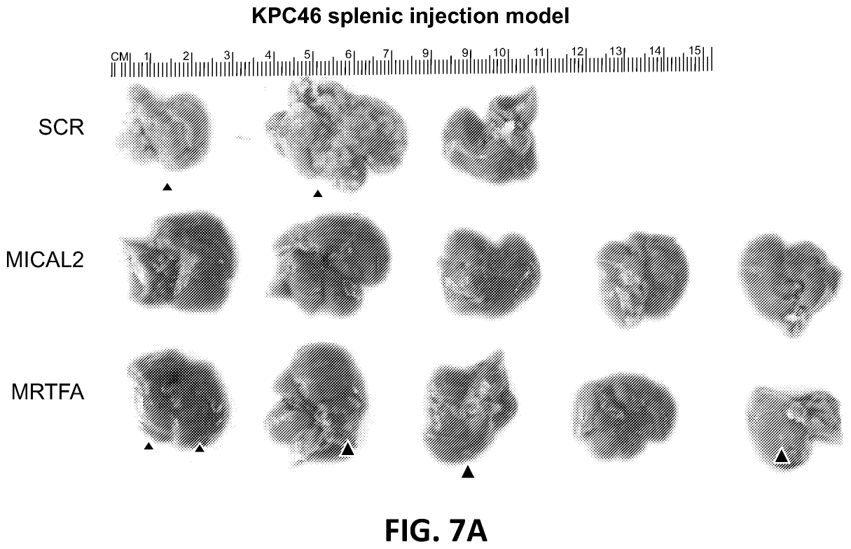

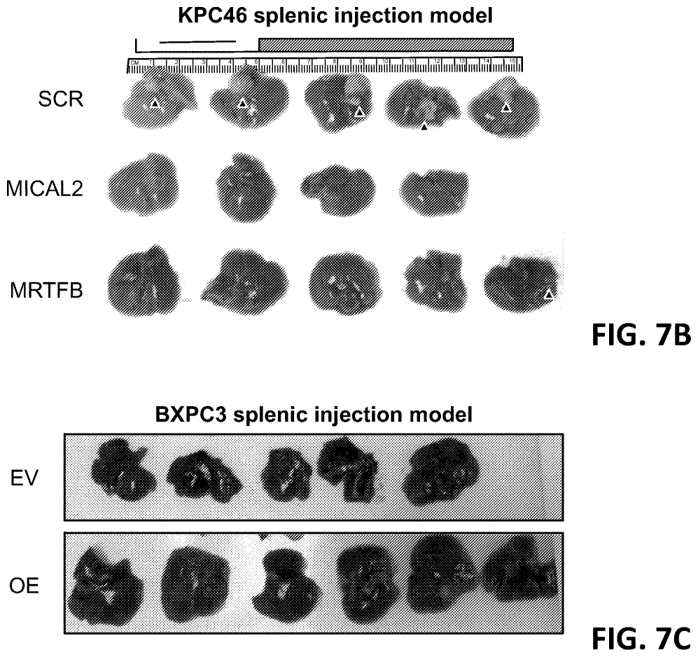

To determine how MICAL2 and MRTF expression impact the competency of PDAC cells to metastasize to the liver, PDAC cells were injected into the spleen of mice. First, the KPC46 cells with KD of MICAL2, MRTF-A, and MRTF-B were injected (FIGS. 7A-7B). Grossly, a dramatic decrease in liver metastatic burden was observed in the MICAL2, MRTF-A and MRTF-B KD models compared to scramble control cells. Histologically, only small, and often merely microscopically detectable liver metastases was found in the KD models compared to extensive gross metastatic disease when the control cells where injected.

Then, BxPc3 cells overexpressing MICAL2 or vector control were injected into the spleen of NSG mice. While gross metastatic disease was not observed in either group, microscopic analysis revealed numerous metastatic foci in livers of mice implanted with BxPc3 MICAL2 overexpressing cells while there was no metastatic disease detectable in the control group (FIG. 7C). These results suggest that MICAL2 expression promotes liver metastasis in PDAC cells.

What is claimed is:

1. A method of treating pancreatic cancer in a subject, the method comprising:
administering to the subject a therapeutically effective amount of a composition comprising an inhibitor of MICAL2, thereby treating the pancreatic cancer in the subject.

2. The method of claim 1, wherein the inhibitor of MICAL2 comprises an inhibitory nucleic acid.

3. The method of claim 2, wherein the inhibitory nucleic acid comprises a small interfering RNA (siRNA), a small hairpin RNA (shRNA), an antisense oligonucleotide (ASO), or a small nuclear RNA (snRNA) targeting a MICAL2 nucleic acid.

4. The method of claim 2, wherein the inhibitory nucleic acid comprises a small hairpin RNA (shRNA).

5. The method of claim 4, wherein the shRNA comprises any one of SEQ ID NOs: 1-3.

6. The method of claim 2, wherein the inhibitory nucleic acid comprises a small interfering RNA (siRNA).

7. The method of claim 6, wherein the siRNA comprises any one of SEQ ID NOs: 4-11.

8. A method of suppressing tumor growth in a subject with cancer, the method comprising:
administering to the subject a therapeutically effective amount of a composition comprising an inhibitor of MICAL2, thereby suppressing tumor growth in the subject.

9. A method of suppressing tumor metastasis in a subject with cancer, the method comprising:
administering to the subject a therapeutically effective amount of a composition comprising an inhibitor of MICAL2, thereby suppressing tumor metastasis in the subject.

10. A method of enhancing response to chemotherapy in a subject with cancer, the method comprising:
administering to the subject a therapeutically effective amount of a composition comprising an inhibitor of MICAL2, thereby enhancing response to chemotherapy in the subject.

11. The methods of any one of claims 8-10, the cancer is a solid tumor.

12. The method of claim 11, wherein the cancer is an appendix cancer, bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

13. The method of claim 11, wherein the cancer is a pancreatic cancer.

14. The method of any one of claims 8-13, wherein the inhibitor of MICAL2 comprises an inhibitory nucleic acid.

15. The method of claim 14, wherein the inhibitory nucleic acid comprises a small interfering RNA (siRNA), a small hairpin RNA (shRNA), an antisense oligonucleotide (ASO), or a small nuclear RNA (snRNA) targeting a MICAL2 nucleic acid.

16. The method of claim 14, wherein the inhibitory nucleic acid comprises a small hairpin RNA (shRNA).

17. The method of claim 16, wherein the shRNA comprises any one of SEQ ID NOs: 1-3.

18. The method of claim 14, wherein the inhibitory nucleic acid comprises a small interfering RNA (siRNA).

19. The method of claim 18, wherein the siRNA comprises any one of SEQ ID NOs: 4-11.

20. The method of any one of claims 1-19, further comprising administering an anti-cancer treatment.

21. The method of claim 20, wherein the anti-cancer treatment comprises surgery, administering ionizing radiation, a chemotherapeutic agent, a therapeutic antibody, a checkpoint inhibitor, or any combination thereof.

22. The method of claim 20, wherein the anti-cancer treatment comprises a chemotherapeutic agent.

23. The method of claim 22, wherein the chemotherapeutic agent comprises vincristine, prednisone, dexamethasone, busulfan, cisplatin, carboplatin, paclitaxel, docetaxel, nab-paclitaxel, altretamine, capecitabine, cyclophosphamide, etoposide (vp-16), gemcitabine, ifosfamide, irinotecan (SN-38), liposomal doxorubicin, melphalan, pemetrexed, topotecan, vinorelbine, goserelin, leuprolide, tamoxifen, letrozole, anastrozole, exemestane, bevacizumab, olaparib, rucaparib, niraparib, nivolumab, pembrolizumab, durvalumab, atezolizumab, radioisotopes, monomethyl auristatin E (MMAE), calicheamicins, deruxtecan, DM1, 5FU, oxaliplatin, and any combinations thereof.

24. The method of claim 22, wherein the chemotherapeutic agent comprises gemcitabine.

25. The method of any one of claims 1-24, wherein the administering comprises intravenous administration, subcutaneous administration, intraperitoneal administration, rectal administration, oral administration, or combinations thereof.

26. A method of modulating MICAL2 expression comprising:
delivering an inhibitory nucleic acid into a cell, wherein the inhibitory nucleic acid comprises any one of SEQ ID NOs: 1-11.

27. The method of claim 26, wherein the inhibitory nucleic acid inhibits MICAL2 by knockdown of the MICAL2 gene expression.

28. A composition comprising an inhibitory nucleic acid of MICAL2, wherein the inhibitory nucleic acid comprises any one of SEQ ID NOs: 1-11.

29. The composition of claim 28, wherein the composition is used in a method of treating pancreatic cancer in a subject, and wherein the composition is administered with an anti-cancer treatment.

30. The composition of claim 29, wherein the anti-cancer treatment comprises surgery, administering ionizing radiation, a chemotherapeutic agent, a therapeutic antibody, a checkpoint inhibitor, or any combination thereof.

31. The composition of claim 29, wherein the anti-cancer treatment comprises a chemotherapeutic agent.

32. The composition of claim 31, wherein the chemotherapeutic agent comprises vincristine, prednisone, dexamethasone, busulfan, cisplatin, carboplatin, paclitaxel, docetaxel, nab-paclitaxel, altretamine, capecitabine, cyclophosphamide, etoposide (vp-16), gemcitabine, ifosfamide, irinotecan (SN-38), liposomal doxorubicin, melphalan, pemetrexed, topotecan, vinorelbine, goserelin, leuprolide, tamoxifen, letrozole, anastrozole, exemestane, bevacizumab, olaparib, rucaparib, niraparib, nivolumab, pembrolizumab, durvalumab, atezolizumab, radioisotopes, monomethyl auristatin E (MMAE), calicheamicins, deruxtecan, DM1, 5FU, oxaliplatin, and any combinations thereof.

33. The composition of claim 31, wherein the chemotherapeutic agent comprises gemcitabine.

34. The method of any one of claims 29-33, wherein the administering comprises intravenous administration, subcutaneous administration, intraperitoneal administration, rectal administration, oral administration, or combinations thereof.
